SEQUENCE LISTING (30 sequences)

SEQ ID NO: 1 - DNA, 18 bp, Artificial Sequence
  TW3 primer
ggtccgtgtttcaagacg

SEQ ID NO: 2 - DNA, 22 bp, Artificial Sequence
  ITS1-f primer
cttggtcatttagaggaagtaa

SEQ ID NO: 3 - DNA, 20 bp, Artificial Sequence
  CIP-pA primer
agagtttgatcatggctcag

SEQ ID NO: 4 - DNA, 20 bp, Artificial Sequence
  CIP-pH primer
aaggaggtgatccaaccgca

SEQ ID NO: 5 - DNA, 17 bp, Artificial Sequence
  M13 (+20) primer
caggaaacagctatgac

SEQ ID NO: 6 - DNA, 16 bp, Artificial Sequence
  M13 (-20) primer
gtaaaacgacggccag

SEQ ID NO: 7 - DNA, 22 bp, Artificial Sequence
  Mala F primer
ctaaatatcggggagagaccga

SEQ ID NO: 8 - DNA, 29 bp, Artificial Sequence
  Mala R primer
gtacttttaactctctttccaaagtgctt

SEQ ID NO: 9 - DNA, 21 bp, Artificial Sequence
  Mala MGB probe
ttcatctttccctcacggtac

SEQ ID NO: 10 - DNA, 21 bp, Artificial Sequence
  Mala probe
ttcatctttccctcacggtac

SEQ ID NO: 11 - DNA, 18 bp, Artificial Sequence
  Mrest-F primer
ggcggccaagcagtgttt

SEQ ID NO: 12 - DNA, 25 bp, Artificial Sequence
  Mrest-R primer
aaccaaacattcctcctttaggtga

SEQ ID NO: 13 - DNA, 17 bp, Artificial Sequence
  Mrest-MGB probe
ttctcctggcatggcat

SEQ ID NO: 14 - DNA, 17 bp, Artificial Sequence
  Mrest probe
ttctcctggcatggcat

SEQ ID NO: 15 - DNA, 15 bp, Artificial Sequence
  Mglob F primer
ggccaagcgcgctct

SEQ ID NO: 16 - DNA, 24 bp, Artificial Sequence
  Mglob R primer
ccacaaccaaatgctctcctacag

SEQ ID NO: 17 - DNA, 18 bp, Artificial Sequence
  Mglob R probe
atcatcaggcatagcatg

SEQ ID NO: 18 - DNA, 18 bp, Artificial Sequence
  Mglob probe
atcatcaggcatagcatg

SEQ ID NO: 19 - DNA, 20 bp, Artificial Sequence
  802F primer
ggaggaacaccrgtggcgaa

SEQ ID NO: 20 - DNA, 23 bp, Artificial Sequence
  862R primer
gcgtgaactaccagggtatctaa

SEQ ID NO: 21 - DNA, 20 bp, Artificial Sequence
  StaphP MGB probe
ctgtaactgacgctgatgtg

SEQ ID NO: 22 - DNA, 20 bp, Artificial Sequence
  Staph P probe
ctgtaactgacgctgatgtg

SEQ ID NO: 23 - DNA, 18 bp, Artificial Sequence
  591F primer
cgagcgttgtccggattt

SEQ ID NO: 24 - DNA, 18 bp, Artificial Sequence
  654R primer
cacttccgacgcgatcaa

SEQ ID NO: 25 - DNA, 18 bp, Artificial Sequence
  Prop615 P MGB probe
cgtaaagggctcgtaggt

SEQ ID NO: 26 - DNA, 18 bp, Artificial Sequence
  Prop615-P probe
cgtaaagggctcgtaggt

SEQ ID NO: 27 - DNA, 21 bp, Artificial Sequence
  PacnF primer
ctaaggagtttttgtgagtgg

SEQ ID NO: 28 - DNA, 19 bp, Artificial Sequence
  Pacn primer
ctttgcacaacaccacgtc

SEQ ID NO: 29 - DNA, 21 bp, Artificial Sequence
  SepiRpob F
gtgatacgtccatgtaatcca

SEQ ID NO: 30 - DNA, 21 bp, Artificial Sequence
  SepiRpob.R
tttgacagctgatgaagagga

(19) United States
(12) Patent Application Publication
(10) Pub. No.: US 2015/0152486 A1
(43) Pub. Date: Jun. 4, 2015

(54) METHOD OF IN VITRO DIAGNOSING A PELLICULAR STATE IN A SUBJECT AND RELATED APPLICATIONS

(71) Applicants: Isabelle Mouyna, Issy Les Moulineaux (FR); Cecile Clavaud, Levallois-Perret (FR); Christiane Bouchier, Paris (FR); Jean-Paul Latge, Issy Les Moulineaux (FR); Lionel Breton, Versailles (FR)

(72) Inventors: Isabelle Mouyna, Issy Les Moulineaux (FR); Cecile Clavaud, Levallois-Perret (FR); Christiane Bouchier, Paris (FR); Jean-Paul Latge, Issy Les Moulineaux (FR); Lionel Breton, Versailles (FR)

(73) Assignee: L'Oreal S.A., Paris (FR)

(21) Appl. No.: 14/414,301

(22) PCT Filed: Jul. 13, 2012
(86) PCT No.: PCT/IB2012/001573

§ 371 Date: Jan. 12, 2015

(51) Int. Cl. C12Q 1/68; G06F 19/00

(52) U.S. Cl. CPC C12Q 1/689; C12Q 1/6895; G06F 19/345; C12Q 2600/16; C12Q 2600/158

(57) ABSTRACT

The present invention relates to a method of diagnosing a pellicular state in a subject, to a screening method of identifying an antidandruff agent, and to kits and nucleic acid arrays useful for said methods.

### INTRODUCTION

The present invention relates to the technical field of dermatology, and covers both cosmetic and therapeutic aspects of dermatology.

More specifically, the present invention relates to an in vitro method of diagnosing a pellicular state in a subject and to a screening method of identifying an active agent for the prevention or treatment of a pellicular state, The present invention is also directed to kits and nucleic acid arrays to be used in said methods.

Dandruff (D) is a scalp condition occurring in up to 50% of the general population, and has been around for centuries despite several available treatments.

Also known as pityriasis simplex capilitii, p. simplex capitis, and p. sicca, the word dandruff is of Anglo-Saxon origin, a combination of ‘tan’ meaning letter′ and ‘drof’ meaning dirty. It is typically characterized by an excessive flaking of the scalp and often causes itching. In an extreme severe form of dandruff observed in about 5% of the population, also known as seborrheic dermatitis (SD), other areas of the body such as the face and/or chest may also be affected and inflammation of the skin in these areas—including the scalp—can be visible. Although not life-threatening, dandruff is relatively inaesthetic and can lead to a loss of self-esteem.

Up to date, the studies conducted to elucidate the origin of this condition have identified the yeast organism Malassezia as the main causal factor. Although other factors are likely to be involved, antidandruff compositions developed so far involve the use of keratolytic agents to soften and dissolve the flakes, of regulators of keratinization, of antifungal or antiseborrheic agents, if needed of anti-inflammatory agents, and combinations thereof. Natural products such as tea tree oil, honey or cinnamic acid are also proposed, usually combined with other synthetic active agents.

However, these agents only relieve at best the signs of dandruff, without leading to a complete remission of the condition after cessation of use. Typically, once a subject affected by dandruff stops using an antifungal preparation, apparent signs of the condition recur and the Malassezia population increases back to its initial level.

There is thus a need to identify novel effective and better adapted treatments of dandruff, but also a need to characterize the degree of severity of a pellicular state in a subject affected by dandruff in order to notably adjust said treatments.

So far, techniques known to assess a pellicular state mainly rely on a visual inspection of the scalp.

The inventors have surprisingly and unexpectedly identified that a specific imbalance of the resident microbial flora of the scalp, involving notably a significant increase in the density of a Malassezia species concomitant to a decrease in the density of a Propionibacterium species, is directly related to the degree of severity of a pellicular state.

The present invention thus provides for the first time an vitro method of diagnosing a pellicular state in a subject, which is highly predictable, sensitive and which displays high predictive values. The invention further provides a screening method of identifying an active agent for the prevention or treatment of said state, as well as kits and nucleic acid arrays for carrying out said methods.

### DETAILED DESCRIPTION OF THE INVENTION

With respect to the use of the different terms throughout the current specification, the following definitions apply.

By “pellicular state” according to the present invention, it is meant an abnormal desquamation, i.e. loss of dead skin cells, of the scalp in a subject. A pellicular state is typically characterized by an excessive loss of skin cells of the scalp, which may be also accompanied by a mild redness and irritation of the skin. Lost cells are generally visible to the naked eye as whitish or greyish flakes (also named dandruffs) which may be shed and fall from the scalp, and are usually composed of clusters of corneocytes. The number of lost cells reflects the degree of severity of the pellicular state, which can be assessed by different methods such as visual scoring (i.e. ASFS also known as adherent scalp flaking scale; Van Abbe, 1964), squamometry or by using corneocyte counts (Piérard-Franchimont et al., 2006; Piérard G E et al., 1995; Piérard G E et al., 1992). An ASFS score above 10 typically characterizes a pellicular state, which may be also associated with a mild-inflammation of the skin if the ASFS score is above 24. In the case of seborrheic dermatitis (SD), the ASFS score is above 24 and the pellicular state is paired with a skin inflammation, redness and intense itching. By “seborrheic dermatitis” (SD) as used herein, it is thus meant a severe form of pellicular state, which can affect not only the scalp but also the face and/or chest of a subject, and which is associated with skin inflammation.

An “antidandruff agent” (or active agent) is, according to the invention, an ingredient active on a pellicular state as defined above, including severe forms thereof such as seborrheic dermatitis (SD).

The term “microbial population” (“microbiota”, “microbial flora”, or “microbial community”) refers to two or more cells of the micro-organisms inhabiting a particular environment. In the context of the present invention, said micro-organisms include bacteria, archaea, microscopic eukaryotes and fungi (such as yeasts) as well as viruses, and preferably bacteria and fungi. The environment refers to the factors, conditions, or influences in which the micro-organisms are situated and which can have an impact on their growth and development, such as on the skin or hair of a subject, more preferably on the skin or hair of the scalp, the face, the chest or even of the whole body of said subject.

Examples of bacteria according to the present invention include, without limitation, bacteria of the Actinobacteria, Firmicutes and Proteobacteria families, more specifically bacteria belonging to the genera Propionibacterium, Microbacterium, Staphylococcus, Streptococcus, Aerococcus, Alloiococcus, Anaerococcus, Finegoldia, Gemella, Granulicatella, Lactococcus, Peptoniphilus, Acinetobacter, Aurantimonas, Bevundimonas, Haemophylus, Methylobacterium, Moraxella, Neisseria, Paracoccus, Pseudomonas, Sphingomonas, and Stenotrophomonas. Propionibacterium species (or sp.) include, among others, the species Propionibacterium acnes (P. acnes) and Propionibacterium granulosum (P. granulosum). Staphylococcus species include, among others, the species Staphylococcus epidermis (S. epidermis), S. auricularis, S. caprae, S. hominis, and S. schleiferi.

Examples of fungi according to the present invention include, without limitation, fungi of the Ascomycota and Basidiomycota families, more specifically fungi belonging to the genus Debaryomyces, Exophylia, Penicillium, Usnea, Cryptococcus, Malassezia, Rhodotolura, Schizophyllum and Strobilirus. The Malassezia genus include about fourteen species, among which Malassezia restricta (or M. restricta), M. globosa, M. sympodialis, M. slooffiae, M. furfur, M. pachydermatis and M. obtusa, and are referred to as Malassezia species in the present invention.

The terms “phylum”, “class”, “order”, “family”, “genus”/“genera” and “species” are well known to the person skilled in the art, and are to be construed according to their conventional meaning in the field of the invention.

The term “density”, or quantitative distribution, refers to the cells number per volume (e.g. cells·ml⁻¹, cells·L⁻¹, etc) or surface unit (e.g. cells·mm⁻², cells·cm⁻²). In the context of the present invention, the density preferably refers to the cells number per surface unit.

According to the following aspects and embodiments of the invention described herein, a “subject” refers to a mammal, preferably a human or an animal, and even more preferably to a human. Even more preferably, said human belongs to a Caucasian population, and advantageously to a European population.

In the context of the invention, when dealing with a scalp, face or chest of a subject or of a model as defined below, it is preferably meant dealing with the scalp, face or chest skin surface.

In a first aspect, the present invention is thus directed to an in vitro method of diagnosing a pellicular state in a subject, comprising the steps of:
a) detecting and quantifying the density of Malassezia sp. and of Propionibacterium sp. of a microbial population sample from a subject;
b) calculating a ratio R between the density of Malassezia sp. and of Propionibacterium sp. present in said sample;
c) comparing said ratio with a value of reference;
   wherein a ratio R superior to said value of reference is indicative of the existence of a pellicular state in said subject.

The ratio R of step b) is thus determined by dividing the value of density of Malassezia sp. by the value of density of Propionibacterium sp. present in the microbial population sample, as follows:

$$R = \frac{density Malassezia sp .}{density Propionibacterium sp .}$$

The value of reference refers to a ratio of density of Malassezia sp. versus the density of Propionibacterium sp. in a healthy subject. This value can been determined according to statistical standards typically used for diagnostic methods (e.g. Altman et al., 1994).

In a preferred embodiment of the invention, the value of reference is equal to 0.008, preferably equal to 0.013, and even more preferably equal to 0.0196.

According to a particular embodiment of the invention, a ratio R superior to a value of 0.490 is indicative of the existence of a severe pellicular state.

A ratio R comprised between 0.008 and 0.490 is thus indicative of a non-severe pellicular state.

According to another particular embodiment of the invention, a ratio R superior to a value of 2.5 is indicative of the existence of a seborrheic dermatitis. More preferably, a ratio R superior to a value of 3, 3.5, 4, 4.5, 5, 5.5, 6, 6.5, 7, 7.5, 8, 8.5, 9, 9.5, 10, 11, 12, 13, 14, 15, 16, 17, 18, 19, 20, 25 or 30 is indicative of the existence of a seborrheic dermatitis.

A ratio R comprised between 0.490 and 2.5 is thus indicative of a severe mild-inflammatory pellicular state.

In particular, the present invention relates to an in vitro method of diagnosing seborrheic dermatitis (SD) in a subject, comprising the steps of:
a) detecting and quantifying the density of Malassezia sp. and of Propionibacterium sp. of a microbial population sample from a subject;
b) calculating a ratio R between the density of Malassezia sp. and of Propionibacterium sp. present in said sample;
c) comparing said ratio with a value of reference of at least 2.5;
   wherein a ratio R superior to said value of reference is indicative of the existence of seborrheic dermatitis in said subject.

In a preferred embodiment, said value is equal to 3, 3.5, 4, 4.5, 5, 5.5, 6, 6.5, 7, 7.5, 8, 8.5, 9, 9.5, 10, 11, 12, 13, 14, 15, 16, 17, 18, 19, 20, 25 and 30. It is understood that the higher the value, the higher the severity of the seborrheic dermatitis (SD).

In a preferred embodiment, Malassezia sp. or Malassezia species comprises, or consists of, Malassezia restricta (M. restricta), M. globosa, M. sympodialis, M. slooffiae, M. furfur, M. pachydermatis and M. obtusa, more preferably comprises, or consists of, M. restricta and M. globosa, and even more preferably Malassezia sp. refers to M. restricta. The inventors have indeed identified M. restricta as the most predominant fungus, and more particularly as the most predominant Malassezia species, present in the microbial population of the scalp of humans.

Preferably, the Propionibacterium sp. or Propionibacterium species of the invention comprises, or consists of, Propionibacterium acnes (P. acnes) and Propionibacterium granulosum (P. granulosum), and more preferably, Propionibacterium sp. designates P. acnes. The inventors have indeed identified P. acnes as the most predominant bacteria, and more particularly as the most predominant Propionibacterium species, present in the microbial population of the scalp of humans.

It is thus an advantageous embodiment of the invention to provide an in vitro diagnostic method as defined above, wherein Malassezia sp. is M. restricta and Propionibacterium sp. is P. acnes.

More preferably, the microbial population sample from said subject is collected from the scalp, face or chest of the subject, and even more preferably from the scalp.

A preferred method for detecting and quantifying the density of the studied micro-organisms according to step a) of the above method is carried out by amplification followed by quantification of a nucleic acid of the Malassezia sp. and of Propionibacterium sp. of the microbial population sample from the scalp of the subject.

As used herein, the terms “nucleic acid”, “nucleic acid sequence”, “nucleotide”, “nucleotide sequence”, which are interchangeable, refer to a precise succession of natural nucleotides (e.g., A, T, G, C and U), corresponding to a single-stranded or double-stranded DNA such as cDNA, genomic DNA, ribosomal DNA or plasmidic DNA, and the transcription product of said DNA, such as RNA.

A nucleic acid according to the invention may be isolated and prepared by any known method including, but not limited to, any synthetic method, any recombinant method, any ex vivo generation method and the like, as well as combinations thereof.

An “amplification” and “quantification” refers to any procedure(s) that are suitable to generate multiple copies of a nucleic acid of interest and to quantify said copies. The polymerase chain reaction (PCR) is by far the most widely used method to amplify a nucleic acid. The amplification and quantification can be also carried out as a single procedure, such as by quantitative PCR (q-PCR) or by FISH (fluorescence in situ hybridization). Examples of quantitative PCR tools are commercially available and include, but are not limited to, Taqman® MGB probes (Applied Biosystems), SYBR® green (Applied Biosystems), Molecular beacons, and Scorpion™ probes (Sigma-Aldrich). In a preferred embodiment of the invention, step a) of the in vitro method of diagnosing is carried out by quantitative PCR.

It shall be understood that, according to the invention, the nucleic acid to be amplified and quantified may be conserved among the genus to be analysed (e.g. among Malassezia sp. or Propionibacterium sp.), or may be specific to the species to be analysed (e.g. M. restricta or P. acnes). By “conserved”, it is meant that the nucleic acid sequence shares a high homology within the genus to be analysed. In general this is meant to construe that said sequence is essentially identical in at least 60%, preferably 70%, 80%, more preferably 90%, even more preferably 97%, 98%, 99% of the known micro-organisms belonging to said genus, and wherein essentially identical means that no more than 8, more preferably no more than 7, 6, 5, 4, 3, 2, or 1 nucleotide is different. In any case, a nucleic acid sequence is considered conserved within the context of the current invention when it is suitable for binding by a probe or primer, thereby allowing to discriminate micro-organisms from one genus from the other, i.e. a probe or a primer is specific for at least one genus when it will not, or essentially not, bind to a substantial part of the sequences of known micro-organisms of another genus, which may be analyzed by a method as defined above (e.g. PCR). By contrast, a nucleic acid sequence is considered specific when it is suitable for binding by a probe or primer, thereby allowing to discriminate micro-organisms from one species from the other within a genus, i.e. a probe or a primer is specific for at least one species when it will not, or essentially not, bind to a substantial part of the sequences of known micro-organisms of the same genus or other genus, which may also be analyzed by a method as defined above (e.g. PCR). According to a preferred embodiment, the nucleic acid sequence to be amplified and quantified according to the invention is specific to the species M. restricta and/or to P. acnes. In a particular embodiment, the nucleic acid sequence to be amplified and quantified is ribosomal DNA (rDNA), such as the bacterial 16S rDNA and the fungal ITS1-5,8S-ITS2-(D1/D2)-28S (ITS-28S) rDNA.

The probes and primers required or useful to carry out the amplification and/or quantification of a nucleic acid of interest are referred to as “nucleic acid fragments” in the context of the invention.

By “nucleic acid fragment”, it is more generally meant herein a nucleic acid hybridizing to a nucleic acid of interest. For instance, such nucleic acid fragment may be at least 10 nucleotides in length or preferably, at least 15 nucleotides in length. They may also be at least 25 or at least 50 nucleotides in length.

In the context of the present invention, the nucleic acid fragment will preferably hybridize to the nucleic acid of interest under stringent hybridization conditions. One example of stringent hybridization conditions is where attempted hybridization is carried out at a temperature from about 50° C. to about 65° C. using a salt solution which is about 0.9 molar. However, the skilled person will be able to vary such conditions in order to take into account variables such as the nucleic acid fragment length, base composition, type of ions present, etc.

A “primer” more specifically refers to a nucleic acid fragment that serves as a starting point for amplification of a nucleic acid of interest. Examples of nucleic primers of the invention include, but are not limited to, the primers of sequence SEQ ID No 7, SEQ ID No 8, SEQ ID No 10, SEQ ID No 11, SEQ ID No 12, SEQ ID No 14, SEQ ID No 15, SEQ ID No 16, SEQ ID No 18, SEQ ID No 19, SEQ ID No 20, SEQ ID No 22, SEQ ID No 23, SEQ ID No 24, SEQ ID No 26, SEQ ID No 27, SEQ ID No 28, SEQ ID No 29, and SEQ ID No 30. Such primers can be used in “a primer set” to amplify the nucleic acid of interest. Examples of primer set of the invention include, but are not limited to, the primer sets (SEQ ID No 7, SEQ ID No 8), (SEQ ID No 11, SEQ ID No 12), (SEQ ID No 15, SEQ ID No 16), (SEQ ID No 19, SEQ ID No 20), (SEQ ID No 23, SEQ ID No 24), (SEQ ID No 27, SEQ ID No 28), and (SEQ ID No 29, SEQ ID No 30).

A “probe” more specifically refers to a nucleic acid fragment that can be used for detection of a nucleic acid of interest. This term encompasses various derivative forms such “fluorescent probe”. When used in combination with a primer set as defined above, said probe can be used for quantification of a nucleic acid of interest. Examples of probes of the invention include, but are not limited to, the probes of sequence SEQ ID No 7, SEQ ID No 8, SEQ ID No 9, SEQ ID No 10, SEQ ID No 11, SEQ ID No 12, SEQ ID No 13, SEQ ID No 14, SEQ ID No 15, SEQ ID No 16, SEQ ID No 17, SEQ ID No 18, SEQ ID No 19, SEQ ID No 20, SEQ ID No 21, SEQ ID No 22, SEQ ID No 23, SEQ ID No 24, SEQ ID No 25 and SEQ ID No 26. Most preferred probes according to the invention include the probes SEQ ID No 10, SEQ ID No 14, SEQ ID No 18, SEQ ID No 22 and SEQ ID No 26. Probes may be labelled by isotopes, radiolabels, binding moieties such as biotin, haptens such as digoxygenin, luminogenic, mass tags, phosphorescent or fluorescent moieties, or by fluorescent dyes alone (e.g., MGB, FAM, VIC, TET, NED, TAMRA, JOE, HEX, ROX, etc) or in combination with other dyes. These labels provide signals detectable by fluorescence, radioactivity, colorimetry, gravimetry, X-ray diffraction or absorption, magnetism, enzymatic activity, mass spectrometry, binding affinity and the like, and facilitate the detection or quantification of the nucleic acid of interest. Examples of probes labelled by fluorescent dyes of the invention include, but are not limited to, the probes of sequence SEQ ID No 9, SEQ ID No 13, SEQ ID No 17, SEQ ID No 21, and SEQ ID No 25.

It is understood that the sequences of nucleic acid fragments as provided herein are expressed in standard IUB/IUPAC nucleic acid code.

In a preferred embodiment of the invention, step a) of the above-described method is carried out for Malassezia sp. by using at least a nucleic acid fragment selected from the group of nucleic acid fragments of sequence SEQ ID No 7, SEQ ID No 8, SEQ ID No 9, SEQ ID No 10, variants thereof and complementary sequences thereof. More preferably, step a) for Malassezia sp. is carried out by using the primer set (SEQ ID No 7, SEQ ID No 8) combined with the probe of sequence SEQ ID No 9.

In a more preferred embodiment of the invention, step a) of the above-described method is carried out for M. restricta by using at least a nucleic acid fragment selected from the group of nucleic acid fragments of sequence SEQ ID No 11, SEQ ID No 12, SEQ ID No 13, SEQ ID No 14, variants thereof and complementary sequences thereof. More preferably, step a) for M. restricta is carried out by using the primer set (SEQ ID No 11, SEQ ID No 12) combined with the probe of sequence SEQ ID No 13.

Yet, in another preferred embodiment of the invention, step a) of the above-described method is carried out for Propionibacterium sp. by using at least a nucleic acid fragment selected from the group of nucleic acid fragments of sequence SEQ ID No 23, SEQ ID No 24, SEQ ID No 25, SEQ ID No 26, variants thereof and complementary sequences thereof. More preferably, step a) for Propionibacterium sp. is carried out by using the primer set (SEQ ID No 23, SEQ ID No 24) combined with the probe of sequence SEQ ID No 25.

In an even more preferred embodiment of the invention, step a) of the above-described method is carried out for P. acnes by using at least a nucleic acid fragment selected from the group of nucleic acid fragments of sequence SEQ ID No 27, SEQ ID No 28, SEQ ID No 25, SEQ ID No 26, variants thereof and complementary sequences thereof. More preferably, step a) for P. acnes is carried out by using the primer set (SEQ ID No 27, SEQ ID No 28) combined with the probe of sequence SEQ ID No 25.

As used herein, the term “complementary” means that, for example, each nucleotide of a first nucleic acid sequence is paired with the complementary base of a second nucleic acid sequence whose orientation is reversed. Complementary nucleotides are A and T (or A and U) or C and G.

“Variants” of a nucleic acid fragment according to the present invention include, but are not limited to, nucleic acid sequences which are at least 99% identical after alignment to said nucleic acid fragment and retain their capacity to hybridize to a nucleic acid of interest as defined above. Examples of variants are degenerate nucleic acid fragments.

Identity between nucleic acid sequences can be determined by comparing a position in each of the sequences which may be aligned for the purposes of comparison. When a position in the compared sequences is occupied by the same nucleotide, then the sequences are identical at that position. A degree of sequence identity between nucleic acids is a function of the number of identical nucleotides at positions shared by these sequences. To determine the percentage of identity between two nucleic acid sequences, the sequences are aligned for optimal comparison. For example, gaps can be introduced in the sequence of a first nucleic acid sequence for optimal alignment with the second nucleic acid sequence. The nucleotides at corresponding nucleotide positions are then compared. When a position in the first sequence is occupied by the nucleotide as the corresponding position in the second sequence, the molecules are identical at that position. The percentage of identity between the two sequences is a function of the number of identical positions shared by the sequences. Hence % identity=number of identical positions/total number of overlapping positions×100. In this comparison, the sequences can be of the same length or may be of different lengths. Optimal alignment of sequences may be conducted by the global homology alignment algorithm of Needleman and Wunsch (1972), by computerized implementations of this algorithm or by visual inspection. The best alignment (i.e., resulting in the highest percentage of identity between the compared sequences) generated by the various methods is selected. In other words, the percentage of sequence identity is calculated by comparing two optimally aligned sequences, determining the number of positions at which the identical nucleotide occurs in both sequences to yield the number of matched positions, dividing the number of matched positions by the total number of positions and multiplying the result by 100 to yield the percentage of sequence identity.

It is another aspect of the invention to provide a method of identifying an antidandruff agent, comprising the steps of:
a) administering a candidate antidandruff agent to a subject or to a dandruff model, wherein a ratio R between the density of Malassezia sp. and of Propionibacterium sp. present on said subject or on said model is superior to the value of reference as defined above;
b) calculating a ratio R′ between the density of Malassezia sp. and of Propionibacterium sp. present on said subject or on said model contacted with said agent;
   wherein a ratio R′ inferior or equal to said value of reference is indicative that said agent is an antidandruff agent.

More preferably, a ratio R′ inferior or equal to 75 percent of said value of reference is indicative that said agent is an antidandruff agent.

The methods for calculating the ratio R′ and for detecting and quantifying the density of micro-organisms are as described above. Advantageous embodiments as mentioned above similarly apply with regard to Malassezia sp. and Propionibacterium sp.

In a preferred embodiment, step a) of administering said candidate agent is carried out for at least 2 to 3 weeks, and more preferably for at least 6 weeks.

By “administering” according to the invention, it is meant delivering or dispensing an agent or composition of interest topically, orally, sublingually, nasally (e.g. aerosol), or by injection such as by subcutaneous injection, intravenous injection, intramuscular injection or intraperitoneal injection. In the context of the invention, step a) of “administering” the candidate antidandruff agent according to the above method is preferably carried out by delivering said agent topically or orally, and more preferably topically.

According to a preferred embodiment, step a) consists in administering said candidate agent to a subject, wherein Malassezia sp. and Propionibacterium sp. are present on the scalp, chest or face of said subject, and even more preferably on the scalp.

According to another preferred embodiment of the invention, step a) consists in administering said candidate agent to a dandruff model, preferably topically.

Examples of dandruff models are known to the person skilled in the art and include, but are not limited to, in vitro models as well as animal models used in laboratories, such as mouse, rabbit, or guinea pig models (Oble et al., 2005; Hewitson et al., 2012; Troller et al., 1971; Ashbee and Bond, 2010). Methods to develop or adapt said models are available to the person skilled in the art.

According to a more preferred embodiment, when step a) is carried out on a dandruff animal model as defined above, the method of identifying said antidandruff agent further comprises step c) of killing said animal.

In particular, the invention is directed to a method of identifying an antidandruff agent, comprising the steps of:
a) administering a candidate antidandruff agent to a subject or to a seborrheic dermatitis model, wherein a ratio R between the density of Malassezia sp. and of Propionibacterium sp. present on said subject or on said model is superior to the value of reference as defined above;
b) calculating a ratio R′ between the density of Malassezia sp. and of Propionibacterium sp. present on of said subject or on said model contacted with said agent;
   wherein a ratio R′ inferior or equal to said value of reference is indicative that said agent is an antidandruff agent.

More preferably, a ratio R′ inferior or equal to 75 percent of said value of reference is indicative that said agent is an antidandruff agent.

According to a preferred embodiment of the invention, step a) consists in administering said candidate agent to a seborrheic dermatitis model, preferably topically.

Examples of seborrheic dermatitis models are known to the person skilled in the art and include, but are not limited to, in vitro models as well as animal models used in laboratories, such as a mouse models of seborrheic dermatitis (e.g. Oble et al., 2005). Methods to develop or adapt said models are available to the person skilled in the art.

According to a more preferred embodiment, when step a) is carried out on a seborrheic dermatitis animal model as defined above, the method of identifying said active agent further comprises step c) of killing said animal.

The methods described above may be performed, for example, by using prepackaged kits or nucleic acid arrays, comprising or consisting of the nucleic acid fragments of the invention.

The invention is thus directed to a kit comprising, or consisting of:
a) at least a nucleic acid fragment hybridizing specifically with a Malassezia sp. nucleic acid; and
b) at least a nucleic acid fragment hybridizing specifically with a Propionibacterium sp. nucleic acid.

According to a preferred embodiment, said nucleic acid fragment hybridizing specifically with a Malassezia sp. nucleic acid is selected from the group consisting of the nucleic acid fragments of sequence SEQ ID No 7, SEQ ID No 8, SEQ ID No 9, SEQ ID No 10, SEQ ID No 11, SEQ ID No 12, SEQ ID No 13, SEQ ID No 14, variants thereof and complementary sequences thereof, more preferably of the nucleic acid fragments of sequence SEQ ID No 7, SEQ ID No 8, SEQ ID No 9, SEQ ID No 10, and even more preferably of the nucleic acid fragments of sequence SEQ ID No 11, SEQ ID No 12, SEQ ID No 13, SEQ ID No 14.

According to another preferred embodiment, said nucleic acid fragment hybridizing specifically with a Propionibacterium sp. nucleic acid is selected from the group consisting of the nucleic acid fragments of sequence SEQ ID No 23, SEQ ID No 24, SEQ ID No 25, SEQ ID No 26, SEQ ID No 27, SEQ ID No 28, variants thereof and complementary sequences thereof, more preferably of the nucleic acid fragments of sequence SEQ ID No 23, SEQ ID No 24, SEQ ID No 25, SEQ ID No 26, and even more preferably of the nucleic acid fragments of sequence SEQ ID No 25, SEQ ID No 26, SEQ ID No 27, SEQ ID No 28.

In a most preferred embodiment, said kit comprises, or consists of:
a) the nucleic acid fragments of sequence SEQ ID No 11, SEQ ID No 12 and SEQ ID No 13; and
b) the nucleic acid fragments of sequence SEQ ID No 27, SEQ ID No 28 and SEQ ID No 25.

The description also discloses a nucleic acid array comprising or consisting of:
a) at least a nucleic acid fragment hybridizing specifically with a Malassezia sp. nucleic acid; and
b) at least a nucleic acid fragment hybridizing specifically with a Propionibacterium sp. nucleic acid.

According to a preferred embodiment, said nucleic acid fragment hybridizing specifically with a Malassezia sp. nucleic acid is selected from the group consisting of the nucleic acid fragments of sequence SEQ ID No 7, SEQ ID No 8, SEQ ID No 9, SEQ ID No 10, SEQ ID No 11, SEQ ID No 12, SEQ ID No 13, SEQ ID No 14, variants thereof and complementary sequences thereof, more preferably of the nucleic acid fragments of sequence SEQ ID No 7, SEQ ID No 8, SEQ ID No 9, SEQ ID No 10, and even more preferably of the nucleic acid fragments of sequence SEQ ID No 11, SEQ ID No 12, SEQ ID No 13, SEQ ID No 14.

According to another preferred embodiment, said nucleic acid fragment hybridizing specifically with a Propionibacterium sp. nucleic acid is selected from the group consisting of the nucleic acid fragments of sequence SEQ ID No 23, SEQ ID No 24, SEQ ID No 25, SEQ ID No 26, SEQ ID No 27, SEQ ID No 28, variants thereof and complementary sequences thereof, more preferably of the nucleic acid fragments of sequence SEQ ID No 23, SEQ ID No 24, SEQ ID No 25, SEQ ID No 26, and even more preferably of the nucleic acid fragments of sequence SEQ ID No 25, SEQ ID No 26, SEQ ID No 27, SEQ ID No 28.

In a most preferred embodiment, said nucleic acid array comprises, or consists of:
a) the nucleic acid fragments of sequence SEQ ID No 11, SEQ ID No 12 and SEQ ID No 13; and
b) the nucleic acid fragments of sequence SEQ ID No 27, SEQ ID No 28 and SEQ ID No 25.

The present invention will be better understood in the light of the following detailed description of experiments, including examples. Nevertheless, the skilled artisan will appreciate that this detailed description is not limitative and that various modifications, substitutions, omissions, and changes may be made without departing from the scope of the invention.

### BRIEF DESCRIPTION OF THE DRAWINGS

FIG. 1. Number of cells per sample detected by qPCR on 20 controls and 29 Dandruff scalps: A) Propionibacterium, Staphylococcus and Malassezia restrcita. B) Ratios M. restricta/Propionibacterium sp. and Staphylococcus sp/Propionibacterium sp. (cells per sample). Ratios were significantly increased in dandruff scalps as compared to non dandruff scalps (p-values=0.004 and 0.0055 respectively). Asterisk indicates a significant statistical difference (p<0.01).

FIG. 2. Intra-individual variation. Box plots comparing microbial communities quantified by qPCR sampled on areas M1 (dandruff area) and M2 (non dandruff area) of the scalp of each individual (D10 to D29). In dandruff scalps M restricta (p=0.0006), Staphylococcus sp. (P=0.002) and the ratio M restricta/Propionibacterium sp. (p=0.03) were increased. Propionibacterium sp. was decreased (p=0.0024).

FIG. 3: Distribution of the bacterial 16S rDNA sequences from 19 subjects (N1-10, controls whiteout dandruff; D1-9 subjects with dandruff.

FIG. 4: Distribution of the fungal ITS-28S rDNA sequences from 19 subjects (N1-10, controls whiteout dandruff; D1-9 subjects with dandruff.

FIG. 5: Distribution of the 4,347 sequences obtained by cloning and sequencing PCR products from the genomic DNA of the 19 subjects from Set-1 and characteristics of the 30 subjects from Set-2.

### EXAMPLES

### I. Experimental Procedures

### I.1. Subject Recruitment and Sample Collection

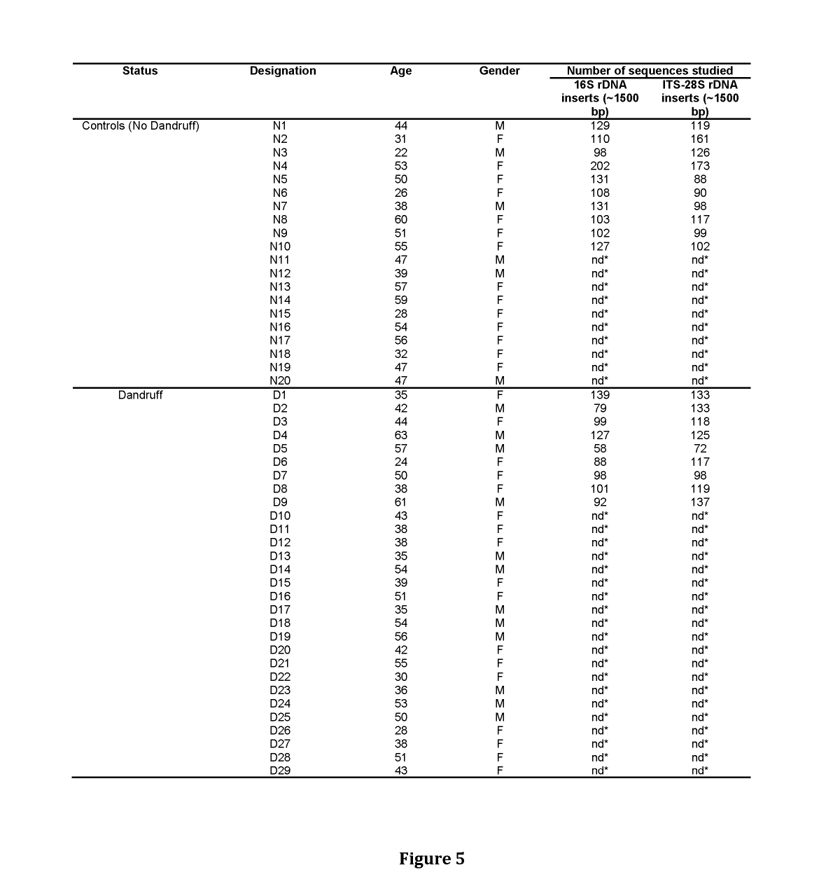

49 healthy adults (22-63 years old; 40% male) were recruited to provide samples and for clinically investigation. Two independent sets of samples were collected and analyzed in this study. Set-1 consisted of samples from 10 non-dandruff and 9 dandruff subjects (subjects N1 to N10 and subjects D1 to D9, respectively) which were analyzed for identification of microbial population followed by qPCR quantification. Set-2 gathered samples from 10 non dandruff and 20 dandruff subjects (i.e. subjects N11 to N20 and subjects D10 to D29, respectively), which were collected in two areas of each scalp: an area with dandruff (M1) and an area without dandruff (M2) (FIG. 5). Human scalps were clinically graded for scalp flaking severity as follows: grades between 0 and 5 were assigned to eight portions of the scalp as compared to reference pictures (0 denoting a region without flakes; 1 to 4.5 denoting low to high amount of flakes; 5 denoting the most flaking region where dandruff covers the entire scalp surface). The final score corresponds to the average of these eight values. Moreover, a grade was assigned to the site of sampling. The study was performed following the Declaration of Helsinki principles and written informed consent was obtained from each subject.

For sample collection, sterile cotton swabs were soaked in 5.0 ml of NaCl 0.15M-Tween20 0.1% buffer. A 16 cm² area was sampled, by passing along 8 lines, four passages per line. The head of each swab was cut from the handle, placed into the tube containing the buffer. Scalp samples were stored at 4° C. and processed for DNA isolation within 24 h. As negative controls, sterile cotton swabs were cut from the handle, placed into 5.0 ml of NaCl 0.15M-Tween20 0.1% buffer and further processed under identical conditions without any contact with scalp.

### I.2. Study Population and Microbial Sequencing

DNA was extracted from scalp samples. For each DNA sample, the bacterial 16S rDNA and the fungal ITS1-5,8S-ITS2-(D1/D2)-28S (ITS-28S) rDNA were PCR-amplified using two sets of universal primers (namely CIP-pA and CIP-pH; TW13 and ITS1f. See Table 1). Fresh PCR products were purified using a PCR purification kit (Qiagen). Clone libraries from both PCR products were sequenced for subject samples from Set1 only and cloned into the pCR2.1-TOPO vector (Invitrogen) and analyzed as described in protocols 1.5 to 1.7 below. After excluding low quality sequences, 2,122 and 2,225 sequences (˜1500 bp) of 16S rDNA and ITS-28S rDNA, respectively, that represented an average of 200 sequences per subject (Clermont et al., 2009). All the sequences were analyzed further at the phylum, genus, and species levels. Taxonomic identity was assigned by Blastn 2.2.21 program in the appropriate reference database. For bacterial identification, the Ribosomal Database Project-II (RDP-II release 10_{—}18, (Cole et al., 2009)) was used while for fungal species found on human skin, a custom database was created with a selection of ITS and 28S fungal sequences of skin fungi (Gemmer et al., 2002; Gao et al., 2007; Paulino et al., 2006; Dekio et al., 2005; Gupta et al., 2004). Sequences were aligned with ClustalW and the maximum likelihood phylogenetic tree was reconstructed with phyml (GTR model).

### I.3. Quantitative PCR

The three major species present in the dandruff environment: P. acnes, S. epidermidis and M. restricta were estimated with Quantitative-PCR by using specific primers and TaqMan MGB probes targeting a species specific region of the bacterial 16S rDNA sequences or the fungal ITS-28S rDNA sequences. For Malassezia genus, M. restricta and M. globosa species, primers and probes reported by Sugita et al. (2010) were used. For S. epidermidis, Staph-P probe (Gao et al., 2006) was used in combination with a set of new primers (802F of sequence SEQ ID No 19, and 862R of sequence SEQ ID No 20; see Table1). For P. acnes, new primers (591 F of sequence SEQ ID No 23, and 654R of sequence SEQ ID No 24) and probe (Prop615-P of sequence SEQ ID No 25) were designed (Table 1). All new primers and probes were designed by using primer express 4 (Applied Biosystem) and the corresponding bacterial 16S rRNA sequences obtained in this study. The standard curves for cell counts were generated for M. restricta, P. acnes and S. epidermidis. To facilitate counting, M. restricta yeasts were dispersed by ultra-sonication. Cell range of concentration between 10² and 10⁷ cells were obtained by 10-fold dilutions before genomic DNA was extracted and used to generate cell standard curve. For real-time PCR, the reaction mix consisted of 20 μl of TaqMan Universal Master Mix II without UNG (Applied Biosystem), 200 nM of each primer (Table 1), 250 nM TaqMan probe (Applied Biosystem) and DNA (0.5-5 ng). Amplification and detection were performed with the iCycler iQ (BIO-RAD) with the following cycle parameters: 55° C. for 2 min, 95° C. for 10 min, and 40 cycles of 95° C. for 30 sec and 55° C. for 30 sec, for Malassezia sp. or 55° C. for 2 min, 95° C. for 10 min, and 40 cycles of 95° C. for 30 sec and 55° C. for 45 sec for bacterial species. Standard curves for each organism were plotted using the cycle threshold (Ct) values. Every sample was run in triplicate.

TABLE 1
Sets of primers used in this study and primers and probes that were
used to quantify M. restricta, M. globose, Propionibacterium sp. and
Staphylococcus sp.

| Primers and probes | Primer sequence (5′-3′) | Reference |
|---|---|---|
| Fungal ITS-28S rDNA | | |
| TW13 (Forward primer) | GGTCCGTGTTTCAAGACG (SEQ ID N^{o} 1) | Taylor et al. (2010) |
| ITS1-f (Reverse primer) | CTTGGTCATTTAGAGGAAGTAA (SEQ ID N^{o} 2) | Taylor et al. (2010) |
| Bacterial 16S rDNA | | |
| CIP-pA (Forward primer) | AGAGTTTGATCATGGCTCAG (SEQ ID N^{o} 3) | Grice et al. (2009) |
| CIP-pH (Reverse primer) | AAGGAGGTGATCCAACCGCA (SEQ ID N^{o} 4) | This study |
| pCR2.1-TOPO vector | | |
| M13 (+20) (Forward primer) | CAGGAAACAGCTATGAC (SEQ ID N^{o} 5) | Invitrogen |
| M13 (−20) (Reverse primer) | GTAAAACGACGGCCAG (SEQ ID N^{o} 6) | Invitrogen |
| All Malassezia species | | |
| Mala-F (Forward primer) | CTAAATATCGGGGAGAGACCGA (SEQ ID N^{o} 7) | Sugita et al. (2006) |
| Mala-R (Reverse primer) | GTACTTTTAACTCTCTTTCCAAAGTGCTT (SEQ ID N^{o} 8) | Sugita et al. (2006) |
| Mala-MGB (probe) | FAM-TTCATCTTTCCCTCACGGTAC-MGB (SEQ ID N^{o} 9) | Sugita et al. (2006) |
| M. restricta | | |
| Mrest-F (Forward primer) | GGCGGCCAAGCAGTGTTT (SEQ ID N^{o} 11) | Sugita et al. (2006) |
| Mrest-R (Reverse primer) | AACCAAACATTCCTCCTTTAGGTGA (SEQ ID N^{o} 12) | Sugita et al. (2006) |
| Mrest-MGB (probe) | FAM-TTCTCCTGGCATGGCAT-MGB (SEQ ID N^{o} 13) | Sugita et al. (2006) |
| M. globose | | |
| Mglob-F (Forward primer) | GGCCAAGCGCGCTCT (SEQ ID N^{o} 15) | Sugita et al. (2006) |
| Mglob-R (Reverse primer) | CCACAACCAAATGCTCTCCTACAG (SEQ ID N^{o} 16) | Sugita et al. (2006) |
| Mglob-MGB (probe) | FAM-ATCATCAGGCATAGCATG-MGB (SEQ ID N^{o} 17) | Sugita et al. (2006) |
| Staphylococcus genus | | |
| 802F (Forward primer) | GGAGGAACACCRGTGGCGAA (SEQ ID N^{o} 19) | This study |
| 862R (Reverse primer) | GCGTGAACTACCAGGGTATCTAA (SEQ ID N^{o} 20) | This study |
| Staph-P (probe) | FAM-CTGTAACTGACGCTGATGTG-MGB (SEQ ID N^{o} 21) | Gao et al., 2010 |
| Propinobacteria genus | | |
| 591F (Forward primer) | CGAGCGTTGTCCGGATTT (SEQ ID N^{o} 23) | This study |
| 654R (Reverse primer) | CACTTCCGACGCGATCAA (SEQ ID N^{o} 24) | This study |
| Prop615-P (probe) | FAM-CGTAAAGGGCTCGTAGGT-MGB (SEQ ID N^{o} 25) | This study |
| Propinobacteria acnes | | |
| PacnF (Forward primer) | CTAAGGAGTTTTTGTGAGTGG (SEQ ID N^{o} 27) | Fenolar et al. (2006) |
| PacnR (Reverse primer | CTTTGCACAACACCACGTC (SEQ ID N^{o} 28) | Fenolar et al. (2006) |
| Staphylococcus epidermis | | |
| SepiRpob.F (Forward primer) | GTGATACGTCCATGTAATCCA (SEQ ID N^{o} 29) | Fenolar et al. (2006) |
| SepiRpob.R (Reverse primer) | TTTGACAGCTGATGAAGAGGA (SEQ ID N^{o} 30) | Fenolar et al. (2006) |

### I.4. Strains and Media

Propionibacterium acnes (strain CIP A179—isolated from sebaceous glands) was kindly provided from the CIP-Library of Institut Pasteur. P. acnes was grown on Medium 20 (tryptone 3%, yeast extract 2%, cysteine hydrochloride 0.05%, glucose 0.5%, agar 1.5% (w/v) and Hemin solution 2,5% (v/v) (Hemin chloride 0.1% (w/v), Triethanolamine 4% (v/v)) pH 7.2) at 37° C. under anaerobic atmosphere for 14 days. Malassezia restricta and Staphylococcus epidermidis strains were isolated from subjects without dandruffs (and characterized by sequencing the 16S rDNA of S. epidermidis and the ITS rDNA of M. restricta). M. restricta cells were grown on Leeming and Notman agar medium (Leeming et al., 1987) at 32° C. for 14 days. S. epidermidis was grown in 2yt medium (bactotryptone 1.6%, bacto yeast extract 1%, sodium chloride 0.5%, and glucose 0.2% pH 7.0) at 37° C. for 24 h under agitation. Cells were then harvested, and counted with haemacytometer.

### I.5. Fungal and Bacterial DNA Extraction

The protocol for DNA isolation is as follows: 2 ml of bacterial-fungal cells suspension from scalp were pelleted by centrifugation for 20 min at 13500 rpm. The collected cells were resuspended in 400 μl of lysis buffer (Tris-HCl 20 mM, NaCl 250 mM, EDTA 25 mM, SDS 1% (w/v), Triton ×100 1% (w/v), pH8.0) containing 5 μl of proteinase K (10 mg/ml, Roche) and incubated for 16 h at 55° C., then for 5 min in a boiling bath. Cells were transferred to screw-capped tubes, 250 μl of glass beads (0.17-0.18 mm, Sartorius) were added and the samples were shaken in a FastPrep (MP Biomedicals) by making three cycles of vortex for 60 s, speed 6.0. Supernatants were further supplemented with phenol and chloroform (400 μl each) and vortexed. After centrifugation for 20 min at 13500 rpm, the aqueous phase was removed and treated with an RNAase (2 μl, 10 mg/ml, Roche) for 3 hours at 37° C. DNA was finally precipitated by adding ammonium acetate (5 mM, final concentration), pure ethanol (1 ml) and resting at −20° C. for 24 h minimum. The resulting DNAs were pelleted by centrifugation (20 min at 13500 rpm) and washed with ethanol 70% (700 μl) and finally suspended in 40 μl of ultra-pure DNAse-RNAse free water. DNA content was measured by Qubit dsDNA HS kit (Invitrogen).

### I.6. PCR Amplification

For each scalp sample, two different PCR products were prepared from genomic DNA. The fungal ITS1-5.8S-ITS2 and part of the 28S (D1/D2) region were amplified with universal primers ITS1f and TW13 (Table 1). 16S rDNA was amplified using the sets of primers: CIP-pA and CIP-pH (Table 1). Both PCR products (˜1,500 bp) were then cloned individually. Amplification method was as follow: in 50 μl reaction mixture, were included 5.0 μl of 10× buffer (Amersham Bioscience), 5.0 μl of dNTP mix (25 mM each; Roche), 1 μl of each primer (10 μM), 1 μl of DMSO, 1 μl of genomic DNA (20 ng) and 0.3 μl of rTaq Polymerase (Amersham) qsp 50 μl of water. For thermocycling, initial denaturation at 95° C. for 5 min, followed by 35 cycles of a 60-sec 95° C. denaturation, 60-sec annealing at 60° C., and 1.5 min elongation at 72° C., all followed by final extension of 15 min at 72° C. PCR products were purified using Qiaquick purification kit (Quiagen) as per manufacturer's instructions.

### I.7. Cloning and Sequencing

Fresh purified PCR products were cloned into the pCR2.1-TOPO vector (Invitrogen) as per manufacturer instructions using 1 μl of PCR product (20 ng DNA). Plasmid DNA purifications were performed using the Montage Plasmid Miniprep96 kit (Millipore). Sequencing reactions were performed with M13 Forward and Reverse primers (of sequence SEQ ID No 5 and SEQ ID No 6, respectively) using ABI Prism BigDye Terminator cycle sequencing-ready reaction kits and run on an ABI 3730 xl Genetic Analyzer. Base calling and quality clipping of the sequence traces were done using the script Assembler Tool Kit.

### I.8. Statistical Methods

ANOVA was used for statistical analysis in this study. The effect of Age, Sex and score on the number of bacterial and fungal cells was tested with Type III Sum of Squares. Normality of the residuals and homogeneity of the variance were visually checked on plots. All the analysis was performed with R program.

More specifically, ratios of the density of each micro-organism respective to each other identified among individuals with and without dandruff were calculated, in order to determine diagnostic values for dandruff based notably on the methods described by Altman D G et al. (1994).

In this regard, the positive predictive value (PPV) for diagnosing a pellicular state in a subject was calculated as:

PPV=number of true positives/(number of true positives+number of false positives),

where a “true positive” is the event that the test makes a positive prediction, and the subject has a positive result under the gold standard, and a “false positive” is the event that the test makes a positive prediction, and the subject has a negative result under the gold standard.

The negative predictive value (NPV) for diagnosing a pellicular state in a subject was calculated as:

NPV=number of true negatives/(number of true negatives+number of false negatives)

where “true negative” is the event that the test makes a negative prediction, and the subject has a negative result under the gold standard, and a “false negative” is the event that the test makes a negative prediction, and the subject has a positive result under the gold standard.

The sensitivity for diagnosing a pellicular state in a subject was calculated as:

sensitivity=number of true positives/(number of true positives+number of false negatives).

And finally, the specificity for diagnosing a pellicular state in a subject was calculated as:

specificity=number of true negatives/(number of true negatives+number of false positives).

### II. Results

II.1. Microbial Diversity on Scalps with and without Dandruff

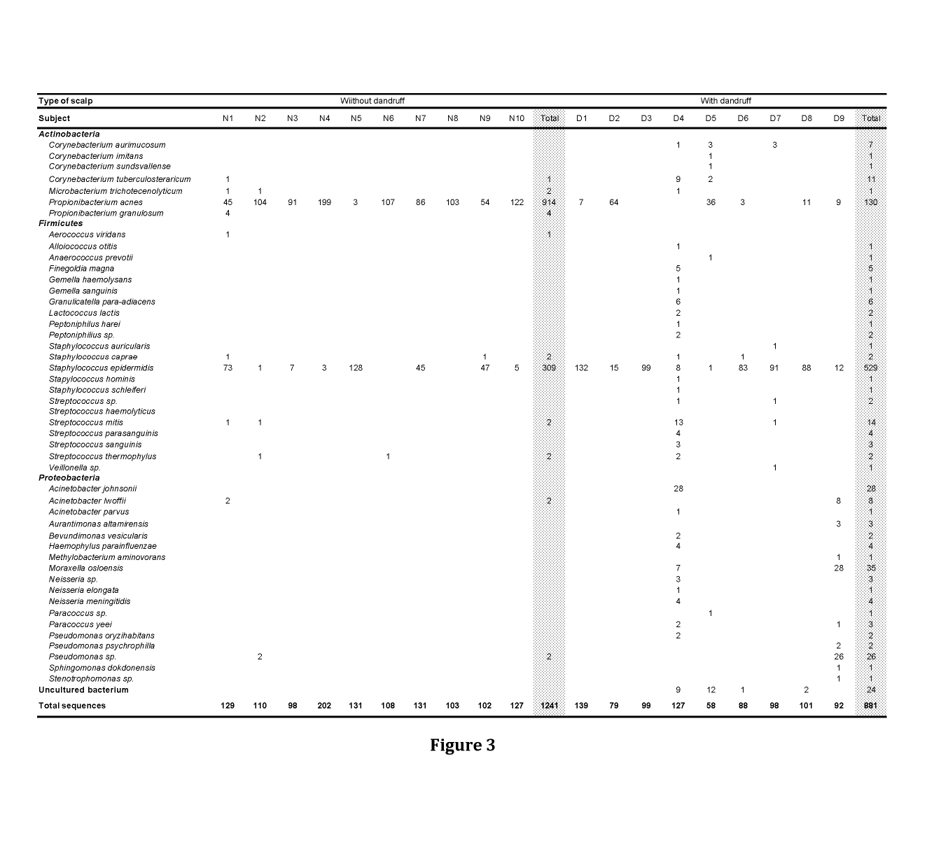

Using Blast analysis, 47 bacterial taxons were identified in the scalps of 19 subjects analysed (9 with dandruff and 10 without, Set 1). Three bacterial phyla: Actinobacteria (51% of the sequences), Firmicutes (42% of the sequences) and Proteobacteria (6% of the sequences) were identified (see FIG. 3). At the genus level, two major genera were observed on the 19 scalp samples including Propionibacteria (49% of all sequences) and Staphylococcus (40% of all sequences), while the remaining 11% of all the sequences included mainly Streptococcus (1.4%), Acinetobacter (1.8%), Corynebacterium (1.0%), Pseudomonas (1.5%) and Moraxella (1.6%). The sequences of Propionibacterium sp. were divided into P. acnes (99.7%) and P. granulosum (0.3%) and the sequences of Staphylococcus sp. were represented mainly by S. epidermidis (99.1%) and S. caprae (0.5%) (FIG. 3).

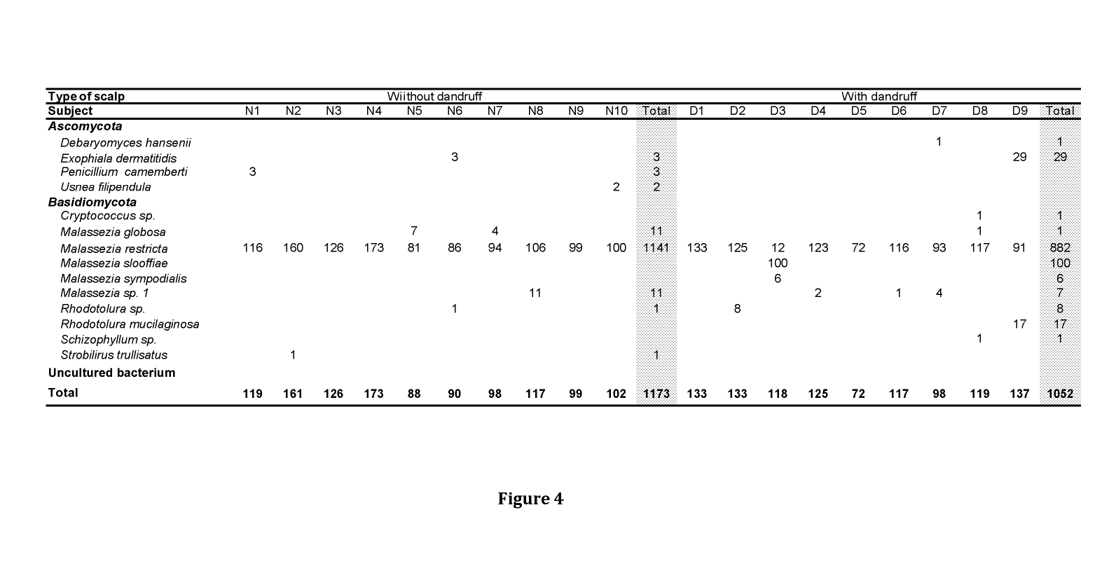

Fourteen fungal taxons were identified in the scalp of the same individuals (FIG. 4). Malassezia restricta was the major fungal species present on the scalps (90% of all sequences) (FIG. 4). Malassezia globosa accounted for less than 1% of all the sequences. We show here that dandruff results from a disequilibrium of the scalp microflora.

### II.2. Unbalance in the Density of Bacterial and Fungal Populations is Linked to the Presence of Dandruff.

Since three main microbial species were only found on the scalp, their quantitative distribution (i.e. density) was analysed among individuals with and without dandruff. The analysis based on quantitative q-PCR was performed on two distinct population samples at two times of the year for a better statistical validation of the results. The PCR primers used for quantification (see Table 1) were specific of the genus Propionobacterium and Staphylococcus but since P. acnes and S. epidermidis counted for 99% of the Propionobacterium and Staphylococcus species identified, respectively, it was considered that the Propionobacterium and Staphylococcus populations quantified corresponded in reality to P. acnes and S. epidermidis, respectively. The 19 subjects from Set-1 and 30 subjects from Set-2 accounted for a total of 29 individuals with dandruff vs 20 controls without dandruff.

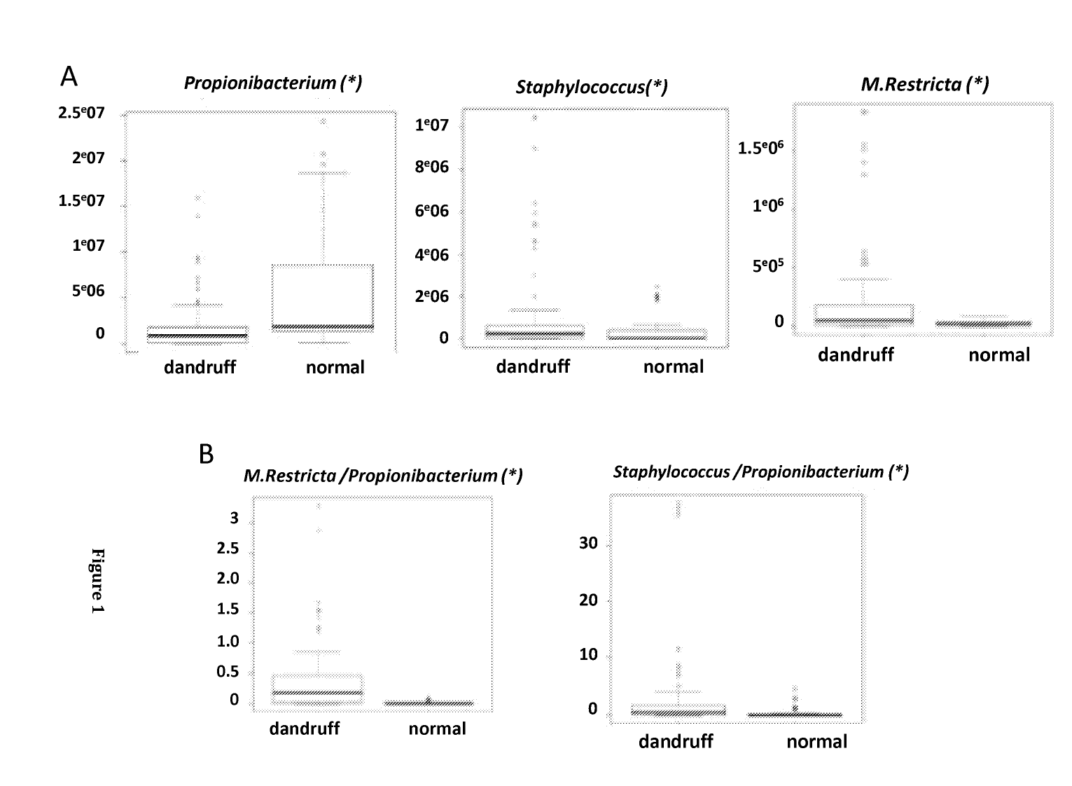

The number of bacterial species detected in all subjects was varying from 1.0 10³ to 1.3 10⁶ (mean value 2.1 10⁵) cells per cm² for Propionibacterium sp and from 5.6 10¹ to 6.2 10⁵ (mean value 4.8 10⁴) cells per cm² for Staphylococcus sp. The number of M. restricta cells ranged from 5.5 10¹ to 1.1 10⁵ (mean value 1.1 10⁴) cells per cm² and accounted for the major part of all Malassezia. Distance-based ANOVA analysis of the differences in the number of bacterial and fungal cells showed a significant decrease in Propionibacterium sp. (p=0.002) (from 3.5 10⁵ to 1.4 10⁵ cells/cm²; p=0.04) and a significant increase in M. restricta (from 1.6 10³ to 1.3 10⁴ cells/cm²; p=0.04), and Staphylococcus sp. (from 2.1 10⁴ to 6.7 10⁴ cells/cm²; p=0.02) in dandruff scalps (FIG. 1A). The proportion between fungal and bacterial populations was investigated. The ratio M. restricta/Propionibacterium sp. was significantly higher (p=0.005) in subjects with dandruff (mean ratio=0.37) as compared to individuals without dandruff (FIG. 1B). In the bacterial communities, the Staphylococcus sp./Propionibacterium sp. ratio was also increased significantly in subjects with dandruff from 0.33 to 2.56 (mean ratios; p=0.004) (FIG. 1B). Variations were independent of age and sex parameters (data not shown). These results were in agreement with the number of sequenced clones specific to each species recovered from each sample (FIGS. 3 and 4).

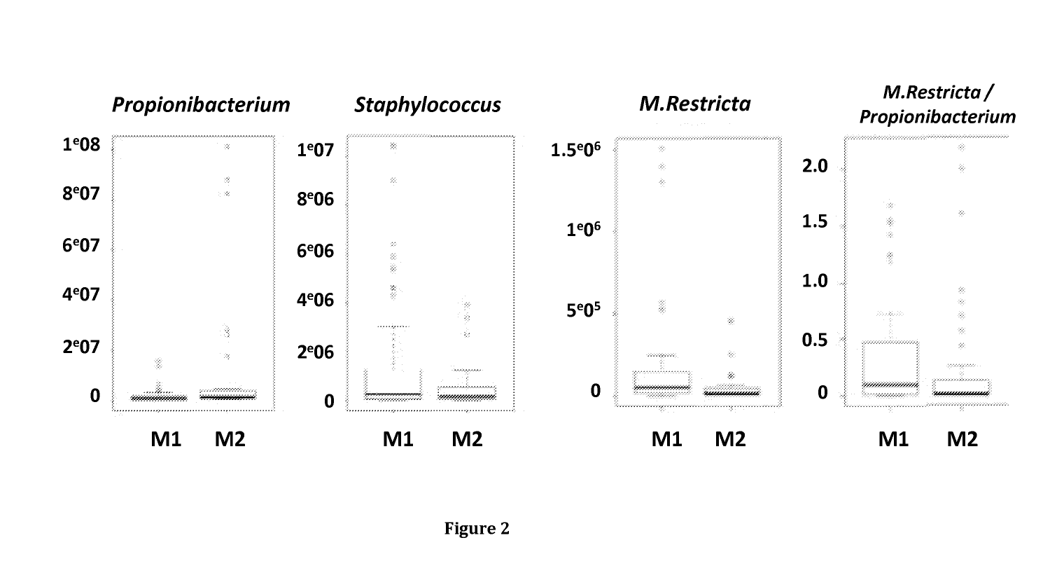

These differences were confirmed within single subjects by comparing the microbial population of an area (M1) with dandruff to an area (M2) without dandruff (FIG. 2). qPCR analysis of the samples collected from M1 and M2 showed similar changes in the microflora as in subjects with dandruff compared to those without dandruff. A significant increase in the quantity of M. restricta (from 1.9 10³ to 7.0 10³ cells/cm²; p=0.0006) and Staphylococcus sp. (from 3.1 10⁴ to 6.7 10⁴ cells/cm²; p=0.002) populations and in M. restricta/Propionibacterium sp. ratio from 0.18 to 0.33 (mean value; p=0.03) was observed, concomitant with a decrease in Propionibacterium sp. (from 3.4 10⁵ to 1.4 10⁵ cells/cm²; p=0.002).

### II.3. Diagnostic Values Related to a Pellicular State

The following diagnostic values have been obtained with regard to the ratio of density of M. restricta versus density of P. acnes present on the scalp of the subjects with and without dandruff.

### II.3.a) X1=0.019

| | Pellicular | Non pellicular |
|---|---|---|
| Positive (R > X1) | VP = 37 | FP = 2 |
| Negative (R < X1) | FN = 12 | VN = 18 |

Test specificity=76%; Test sensitivity=90%; Positive predictive value: 95%; Negative predictive value: 60%.

### II.3.b) X1=0.013

| | Pellicular | Non pellicular |
|---|---|---|
| Positive (R > X1) | VP = 39 | FP = 4 |
| Negative (R < X1) | FN = 10 | VN = 16 |

Test specificity=80%; Test sensitivity=80%; Positive predictive value: 91%; Negative predictive value: 62%.

### II.3.c) X1=0.008

| | Pellicular | Non pellicular |
|---|---|---|
| Positive (R > X1) | VP = 42 | FP = 6 |
| Negative (R < X1) | FN = 7 | VN = 14 |

Test specificity=85%; Test sensitivity=70%; Positive predictive value: 88%; Negative predictive value: 66%.

### II.3.c) X1=0.005

| | Pellicular | Non pellicular |
|---|---|---|
| Positive (R > X1) | VP = 45 | FP = 11 |
| Negative (R < X1) | FN = 4 | VN = 9 |

Test specificity=92%; Test sensitivity=45%; Positive predictive value: 80%; Negative predictive value: 70%.

### II.4. Discussion

Three major species have been found in dandruff or non-dandruff scalps: P. acnes, S. epidermidis and M. restricta. The presence of these two major bacterial species has been reported before on human skin microbiota (Grice et al., 2009), where P. acnes was shown to be predominant in sebaceous rich body sites.

The present data shows that in contrast to other studies, dandruff is not due to the higher incidence of one fungal species but rather to changes in the fungal and bacterial species on the scalp. Although earlier reports suggested the presence of M. restricta and M. globosa associated with dandruff, our study has shown that M. restricta is the most predominant Malassezia species. Real-time PCR quantifications in patients with atopic dermatitis, seborrheic dermatitis, or psoriasis also showed that M. restricta was the most abundant species on the scalp.

Previous reports on the presence of Malassezia sp. in skin lesions demonstrated that differences in lesional skin and healthy skin exhibited the same species, but sub-phylotype specific for lesions could exist. In the current analysis of dandruff species, we also identified P. acnes, S. epidermidis and M. restricta. The comparison of the 824 sequences of S. epidermidis using clustalW showed the presence of two main phylotypes, at a frequency of 44% and 53% respectively. The comparison of the 1005 sequences of P. acnes also showed that two main phylotypes present at frequency of 40 and 50%, respectively. Using the 2000 ITS-28S rDNA sequences of Malassezia restricta obtained by sequencing and phylogenetic analysis, we found that two phylotypes of M. restricta were present. None of the fungal or bacterial phylotypes were preferentially associated with drandruff since the same proportion of each phylotype was found in the dandruff and non-dandruff groups (data not shown).

### II.5. Conclusion

The inventors have demonstrated that dandruff is correlated with an increase in the density of a Malassezia species and S. epidermis, and a decrease in density of P. acnes. They have more specifically identified the ratios between said micro-organisms which characterize a pellicular state, as well as a severe pellicular state, thereby providing tools for better management of this skin condition.

### REFERENCES

Van Abbe N J (1964). J Soc Cosmetic Chem; 15:609-30.
Piérard-Franchimont C, Xhauflaire-Uhoda E, Piérard GE (2006). Int J Cosmet Sci 28:311-8.
Pierard G E, Pierard F C (1995). Skin Res Technol; 1:137-9.
Pierard G E, Pierard F C, Saint-Leger D, Kligman A M (1992). J Soc Cosmet Chem; 47:297-305. Altman D G, Bland J M (1994). British Medical Journal; 309: 102.1 and 308: 1552.
Oble D A, Collett E, Hsieh M, Ambjørn M, Law J, Dutz J, Teh H S (2005). J Invest Dermatol; 124(1):151-9.
Hewitson K S, Vullo D, Scozzafava A, Mastrolorenzo A, Supuran C T (2012). J Med Chem.; 55(7):3513-20.
Troller J A (1971). J Soc Cosmet Chem; 22:187-198.
Ashbee R H and Bond R (2010). Malassezia and the skin, science and clinical practice; chapter 5, p. 139-173, Springer edition. Clermont D, Diard S, Bouchier C et al. (2009). Int J Syst Evol Microbiol; 59:1016-1022.
Cole J R, Wang Q, Cardenas E et al. (2009). Nucleic Acids Res; 37:D141-D145.
Gemmer C M, Deangelis Y M, Theelen B et al. (2002). J Clin Microbiol; 40:3350-3357.
Gao Z, Tseng C H, Pei Z et al. (2007). Proc Natl Acad Sci USA; 104:2927-2932.
Paulino L C, Tseng C H, Strober B E et al. (2006). J Clin Microbiol; 44:2933-2941.
Dekio I, Hayashi H, Sakamoto M et al. (2005). J Med Microbiol; 54:1231-1238.
Gupta A K, Boekhout T, Theelen B et al. (2004). J Clin Microbiol; 42:4253=4260.
Sugita T, Tajima M, Tsubuku H et al. (2006). Microbiol Immunol 50:549-552.
Gao Z, Perez-Perez G I, Chen Y et al. (2010). J Clin Microbiol; 48:3575-3581.
Fenollar F, Roux V, Stein A, Drancourt M, Raoult D (2006). J Clin Microbiol.; March; 44(3):1018-28.
Taylor D L, Booth M G, McFarland J W et al. (2008). Mol Ecol Resour, 8:742-7520.
Grice E A, Kong H H, Conlan S et al. (2009). Science; 324:1190-1192.
Leeming J P and Notman F H (1987). J Clin Microbiol; 25:2017-2019.

1. An in vitro method of diagnosing a pellicular state in a subject, comprising the steps of:
a. detecting and quantifying the density of Malassezia sp. and of Propionibacterium sp. of a microbial population sample from a subject;
b. calculating a ratio R between the density of Malassezia sp. and of Propionibacterium sp. present in said sample;
c. comparing said ratio with a value of reference;
wherein a ratio R superior to said value of reference is indicative of the existence of a pellicular state in said subject.

2. The method according to claim 1, wherein said Malassezia sp. is Malassezia restricta.

3. The method according to claim 1, wherein said Propionibacterium sp. is Propionibacterium acnes.

4. The method according to claim 1, wherein said value of reference is equal to 0.008, preferably to 0.013, and more preferably to 0.0196.

5. The method according to claim 1, wherein said value of reference is equal to 0.490, and wherein a ratio R superior to 0.490 is indicative of the existence of a severe pellicular state.

6. The method according to claim 1, wherein said value of reference is equal to 2.5, and wherein a ratio R superior to 2.5 is indicative of the existence of seborrheic dermatitis.

7. The method according to claim 1, wherein step a) is carried out by quantitative PCR.

8. The method according to claim 7, wherein said quantitative PCR is carried out for Malassezia sp. by using at least a nucleic acid fragment selected from the group of nucleic acid fragments of SEQ ID NO:7, SEQ ID NO:8, SEQ ID NO:9, SEQ ID NO:10, variants thereof, and complementary sequences thereof.

9. The method according to claim 7, wherein said quantitative PCR for Malassezia restricta is carried out by using at least a nucleic acid fragment selected from the group of nucleic acid fragments of SEQ ID NO:11, SEQ ID NO:12, SEQ ID NO:13, SEQ ID NO:14, variants thereof, and complementary sequences thereof.

10. The method according to claim 7, wherein said quantitative PCR for Propionibacterium sp. is carried out by using at least a nucleic acid fragment selected from the group of nucleic acid fragments of SEQ ID NO:23, SEQ ID NO:24, SEQ ID NO:25, variants thereof, and complementary sequences thereof.

11. The method according to claim 7, wherein said quantitative PCR for P. acnes. is carried out by using at least a nucleic acid fragment selected from the group of nucleic acid fragments of SEQ ID NO:27, SEQ ID NO:28, SEQ ID NO:25, variants thereof, and complementary sequences thereof.

12. A method of identifying an antidandruff agent, comprising the steps of:
a. administering a candidate antidandruff agent to a subject or to a dandruff model, wherein a ratio R between the density of Malassezia sp. and of Propionibacterium sp. present on said subject or on said model is superior to the value of reference as defined in claim 1;
b. calculating a ratio R′ between the density of Malassezia sp. and of Propionibacterium sp. present on said subject or on said model contacted with said agent;
wherein a ratio R′ inferior or equal to said value of reference is indicative that said agent is an antidandruff agent.

13. A kit for use in a method according to claim 1, comprising:
a. at least a nucleic acid fragment hybridizing specifically with a Malassezia sp. nucleic acid; and
b. at least a nucleic acid fragment hybridizing specifically with a Propionibacterium sp. nucleic acid.

14. The kit for use according to claim 13, wherein:
a. said nucleic acid fragment hybridizing specifically with a Malassezia sp. nucleic acid is selected from the group consisting of the nucleic nucleic acid fragments of SEQ ID NO:7, SEQ ID NO:8, SEQ ID NO:9, SEQ ID NO:10, SEQ ID NO:11, SEQ ID NO:12, SEQ ID NO:13, SEQ ID NO:14, variants thereof, and complementary sequences thereof; and
b. said nucleic acid fragment hybridizing specifically with a Propionibacterium sp. nucleic acid is selected from the group consisting of the nucleic acid fragments of SEQ ID NO:23, SEQ ID NO:24, SEQ ID NO:25, SEQ ID NO:26, SEQ ID NO:27, SEQ ID NO:28, variants thereof, and complementary sequences thereof.

15. A nucleic acid array for use in a method according to claim 1, comprising:
a. at least a nucleic acid fragment hybridizing specifically with a Malassezia sp. nucleic acid; and
b. at least a nucleic acid fragment hybridizing specifically with a Propionibacterium sp. nucleic acid.

16. The nucleic acid array according to claim 15, wherein:
a. said nucleic acid fragment hybridizing specifically with a Malassezia sp. nucleic acid is selected from the group consisting of the nucleic acid fragments of SEQ ID NO:7, SEQ ID NO:8, SEQ ID NO:9, SEQ ID NO:10, SEQ ID NO:11, SEQ ID NO:12, SEQ ID NO:13, SEQ ID NO:14, variants thereof, and complementary sequences thereof; and
b. said nucleic acid fragment hybridizing specifically with a Propionibacterium sp. nucleic acid is selected from the group consisting of the nucleic acid fragments of SEQ ID NO:23, SEQ ID NO:24, SEQ ID NO:25, SEQ ID NO:26, SEQ ID NO:27, SEQ ID NO:28, variants thereof, and complementary sequences thereof.

17. The method according to claim 1, wherein said value of reference is equal to 0.013.

18. The method according to claim 1, wherein said value of reference is equal to 0.0196.
